(19) United States
(12) Patent Application Publication
(10) Pub. No.: US 2024/0428902 A1
(43) Pub. Date: Dec. 26, 2024

(54) INFORMATION PROCESSING DEVICE AND INFORMATION PROCESSING METHOD

(71) Applicant: OMRON Corporation, Kyoto-shi, KYOTO (JP)

(72) Inventor: Shoya MURAKAMI, Kyoto-shi, KYOTO (JP)

(21) Appl. No.: 18/703,560

(22) PCT Filed: Oct. 26, 2022
(86) PCT No.: PCT/JP2022/039982

§ 371 Date: Apr. 22, 2024

(30) Foreign Application Priority Data
Nov. 16, 2021 (JP) 2021-186076

(51) Int. Cl. G16H 10/20; G06V 40/16

(52) U.S. Cl. CPC G16H 10/20; G06V 40/161

(57) ABSTRACT

An information processing device includes an acquisition unit configured to acquire information for determining psychology as being information for determining a psychological state of a user, a determination unit configured to determine the psychological state of the user on the basis of the information for determining psychology, a storage unit configured to store past psychological states of the user in association with pieces of work information, each of the pieces of work information including work content and an amount of work of the user in each of the past psychological states, and a decision unit configured to acquire work information associated with a past psychological state being the same as a current psychological state of the user from the storage unit and decide work content to be presented to the user on the basis of the work information acquired.

### TECHNICAL FIELD

The present invention relates to an information processing device and an information processing method.

### BACKGROUND ART

In a workplace or the like, workers may not be able to communicate well with those around them even when they have a physical or mental illness. When a manager assigns a task without understanding illness of the worker, work content and an amount of work assigned to the worker may not be appropriate for a health state and a psychological state of the worker. Patent Document 1 discloses a technique for generating a work schedule on the basis of a skill of the worker and biological information such as body temperature.

### CITATION LIST

### Patent Literature

Patent Document 1: JP 2019-061530 A

### SUMMARY OF INVENTION

### Technical Problem

Even when the work schedule is generated on the basis of measurable information such as the biological information, when the psychological state of the worker is not taken into consideration, work load on the worker with physical and mental illness may increase and the psychological state of the worker may deteriorate.

In one aspect of the present invention, an object is to present work in consideration of a psychological state of a worker so that the worker can work comfortably.

### Solution to Problem

To achieve the above-mentioned object, the present invention adopts the following configurations.

According to an aspect of the present disclosure, an information processing device includes an acquisition unit configured to acquire information for determining psychology as being information for determining a psychological state of a user, a determination unit configured to determine the psychological state of the user on the basis of the information for determining psychology, a storage unit configured to store past psychological states of the user in association with pieces of work information, each of the pieces of work information including work content and an amount of work of the user in each of the past psychological states, and a decision unit configured to acquire work information associated with a past psychological state being the same as a current psychological state of the user from the storage unit and decide work content to be presented to the user on the basis of the work information acquired.

The information for determining psychology is information for determining the psychological state of the user, such as a captured image of a face of the user, a voice of the user, and an answer to a questionnaire regarding a psychological state. The work content to be presented is the work content to be presented to the user on the basis of the past work information in the same psychological state as the determined psychological state of the user. The information processing device can decide the work content to be presented on the basis of, for example, preset work priorities, changes in the amount of work due to different psychological states, or the like from the pieces of work information acquired. Since the work is presented in consideration of the psychological state of the worker, the worker can work comfortably.

Each of the pieces of work information associated with the past psychological states may further include information on a post-work psychological state after the user performed work content of each of the pieces of work information, and the decision unit may decide the work content to be presented on the basis of work information in which the post-work psychological state is improved compared to a pre-work psychological state from pieces of work information associated with the past psychological state being the same as the current psychological state. The work through which the psychological state improved after the work is presented with priority, making the work more efficient and allowing the worker to work more comfortably.

Each of the pieces of work information associated with the past psychological states may further include information on a psychological state after work presentation when the user is presented with work content of each of the pieces of work information, and the decision unit may decide the work content to be presented on the basis of work information in which the psychological state after work presentation is improved compared to a psychological state before work presentation from pieces of work information associated with the past psychological state being the same as the current psychological state. The work that improved the psychological state after the work content was presented in the past is presented with priority, making the work more efficient and allowing the worker to work more comfortably.

A score may be set for a psychological state one by one for the psychological state of the user, and whether the psychological state of the user improved may be determined in accordance with a change in the score. The information processing device can easily determine whether the psychological state has improved.

When there are multiple pieces of work information associated with the past psychological state being the same as the current psychological state, the decision unit may decide the work content to be presented on the basis of at least one of a degree of improvement in the psychological state of the user or reliability of determination of the psychological state of the user after improvement. The decision unit can accurately decide whether the psychological state has improved and decide the work content to be presented.

The information for determining psychology may include a captured image of a face or part of the face of the user, and the determination unit may determine the psychological state of the user using a learned model trained using the captured image of the face or the part of the face and a psychological state when photographing as training data. The determination unit can determine the psychological state of the user from facial expressions and the like. In addition, by making the determination on the basis of the captured image of the parts of the face in which the psychological state is likely to be expressed, the determination unit can capture a subtle change in a facial expression and more accurately determine the psychological state of the user.

The determination unit may identify the user by face recognition, and determine the psychological state of the user using a learned model trained using captured images of a face of the user or part of the face of the user and psychological states when photographing as training data. By using the learned model trained using the captured image of the user himself or herself identified by the face recognition, the determination unit can more accurately determine the psychological state of the user.

The information for determining psychology may include information indicating a positional relationship between a face and a hand of the user, and the determination unit may determine the psychological state of the user using a learned model trained using the information indicating the positional relationship between the face and the hand of the user and a psychological state when photographing as training data. The determination unit can more accurately determine the psychological state of the user on the basis of a habit of the user indicated by the positional relationship between the face and the hand.

The determination unit may detect the face of the user from a captured image captured with the face positioned within a frame displayed on a display unit by the user. The determination unit can omit processing of detecting the face from the captured image.

The information for determining psychology may include voice data of a voice of the user, and the determination unit may determine the psychological state of the user using a learned model trained using the voice data of the voice of the user and a psychological state when recording as training data. Even when the information processing device is not provided with a camera, the determination unit can determine the psychological state of the user on the basis of the voice data of the voice of the user.

The information for determining psychology may include an answer of the user to a questionnaire for determining the psychological state of the user, and the determination unit may determine the psychological state of the user on the basis of the answer to the questionnaire. Even when it is difficult to determine the psychological state from the facial expression or tone of the voice, the determination unit can more accurately determine the psychological state of the user.

When determining the psychological state of the user using multiple pieces of the information for determining psychology, the determination unit may determine, as the psychological state of the user, a psychological state having the highest reliability from psychological states determined from the multiple pieces of the information for determining psychology. The reliability is reliability when determining the psychological state of the user using a predetermined learned model corresponding to the acquired information for determining psychology. The determination unit can accurately determine the psychological state of the user by adopting the psychological state with higher reliability.

The information processing device may include a presentation unit configured to present the work content to be presented decided by the decision unit to the user. The user can confirm his or her work before starting work. The work content presented to the user may be presented to the manager. By grasping the work content of each user, the manager can manage the overall work schedule.

The information processing device may include an output unit configured to cause the storage unit to store the psychological state of the user determined by the determination unit in association with work information including the work content to be presented decided by the decision unit and an amount of work performed by the user. The information on the psychological state and the corresponding work information are accumulated in the storage unit, enabling the information processing device to present an appropriate work according to the psychological state of the user.

According to another aspect of the present disclosure, an information processing method executed by a computer, the information processing method includes acquiring information for determining psychology as being information for determining a psychological state of a user, determining the psychological state of the user on the basis of the information for determining psychology, and acquiring, from a storage unit configured to store past psychological states of the user in association with pieces of work information, each of the pieces of work information including work content and an amount of work of the user in each of the past psychological states, work information associated with a past psychological state being the same as a current psychological state of the user, and deciding work content to be presented to the user on the basis of the work information acquired.

According to yet another aspect of the present disclosure, there is provided a program causing a computer to function as each unit of the above-described information processing device, or a computer-readable storage medium storing the program in non-transitory.

### Advantageous Effects of Invention

According to the present invention, work can be presented in consideration of a psychological state of a worker, so that the worker can work comfortably.

### BRIEF DESCRIPTION OF THE DRAWINGS

FIG. 1 is a diagram illustrating an application example and a hardware configuration example of an information processing device.

FIG. 2 is a diagram illustrating a functional configuration of the information processing device.

FIG. 3 is a flowchart illustrating work presentation processing according to a first embodiment.

FIG. 4 is a diagram illustrating data in a work information storage unit according to the first embodiment.

FIG. 5 is a flowchart illustrating work presentation processing according to a second embodiment.

FIG. 6 is a diagram illustrating data in a work information storage unit according to the second embodiment.

FIG. 7 is a flowchart illustrating work presentation processing according to a third embodiment.

FIG. 8 is a diagram illustrating data in a work information storage unit according to the third embodiment.

FIG. 9 is a flowchart illustrating processing to acquire information for determining psychology according to Variation 4-1.

FIG. 10 is a diagram illustrating parts of a face according to Variation 4-2.

FIG. 11 is a flowchart illustrating processing to acquire information for determining psychology according to Variation 4-2.

FIG. 12 is a diagram illustrating a positional relationship between a face and a hand according to Variation 4-3.

FIG. 13 is a flowchart illustrating processing to acquire information for determining psychology according to Variation 4-3.

FIG. 14 is a flowchart illustrating processing to acquire information for determining psychology according to Variation 4-4.

FIG. 15 is a diagram illustrating a questionnaire according to Variation 4-5.

FIG. 16 is a flowchart illustrating processing to acquire information for determining psychology according to Variation 4-5.

### DESCRIPTION OF EMBODIMENTS

Embodiments according to one aspect of the present invention will be described below on the basis of the drawings.

### <Application Example>

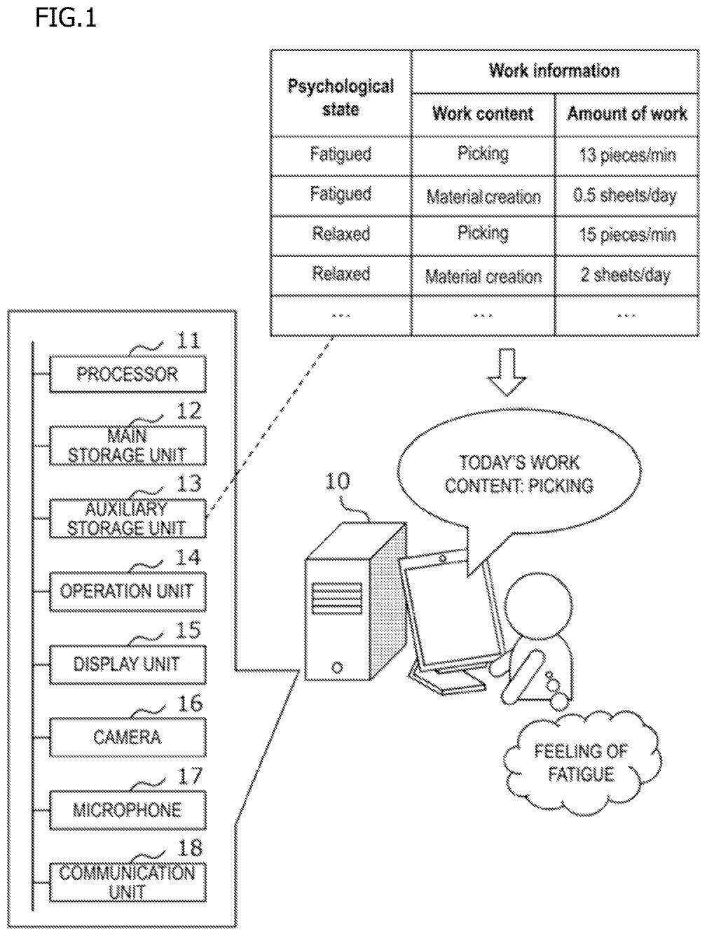

With reference to FIG. 1, an application example of an information processing device that determines a psychological state of a user who is a worker and presents work content according to the psychological state will be described. The information processing device acquires information for determining the psychological state of the user (hereinafter, also referred to as information for determining psychology) such as a captured image of a face of the user, a voice of the user, and answers to a questionnaire regarding the psychological state. The information processing device determines the psychological state of the user on the basis of the acquired information for determining psychology. For example, the information processing device can determine the psychological state by preparing a learned model trained using captured images of the face of the user and psychological states when photographing as training data, and inputting the captured image of the face of the user into the learned model.

The information processing device decides work content according to the determined psychological state of the user and presents the work content to the user. The information processing device includes a storage unit that stores, for each user, a past psychological state in association with information on work content and an amount of work (hereinafter, also referred to as work information) performed in the past psychological state. The information processing device can decide work content to be presented to the user (hereinafter, also referred to as work content to be presented) on the basis of past work information in the same psychological state as the determined psychological state of the user.

When there are multiple pieces of past work information corresponding to the current psychological state of the user, the information processing device can decide the work content to be presented on the basis of work priorities, changes in the amount of work due to deterioration of the psychological state, or the like. The work priorities may be set in advance for each user, for example, when the manager plans an overall work schedule, taking into consideration of influence on the work due to a skill or the psychological state of the user. The information processing device may decide the work content to be presented by giving a priority to work with a value that exceeds a standard value of the amount of work (e.g., an average value of the amount of work when multiple other users work in the same psychological state), that is, work that requires a higher skill than that required for the multiple other users.

FIG. 1 illustrates an example in which it is determined that the user has a feeling of fatigue. An amount of work for material creation is significantly lower than in a relaxed situation, so the information processing device presents “picking”, which has a lower degree of reduction of the amount of work than “material creation”, as today's work content.

In this way, the information processing device can determine the psychological state of the user and present work content according to the current psychological state of the user on the basis of the work content and the amount of work performed in the past in the same psychological state. Thus, the user can work comfortably in accordance with his or her psychological state. In addition, once the psychological state of the user is determined, the manager can grasp the amount of work in the determined psychological state from the past work information, making it easier to manage the work schedule.

### First Embodiment

### (Hardware Configuration)

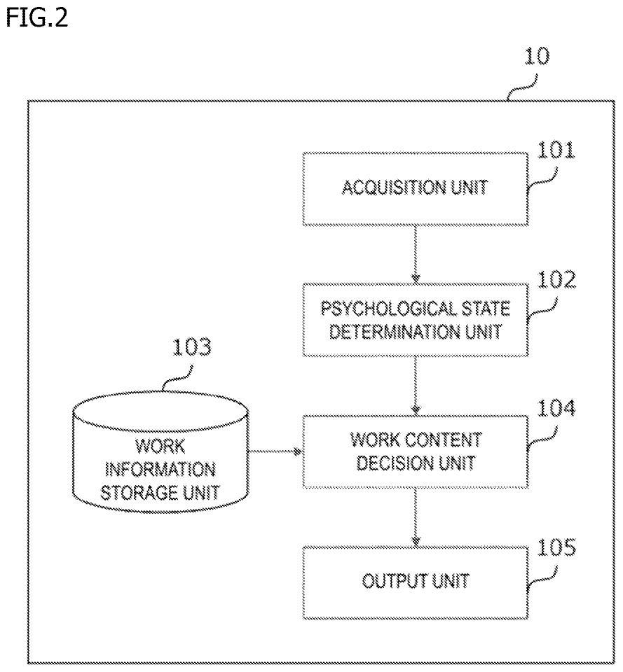

With reference to FIG. 1, an example of a hardware configuration of an information processing device 10 will be described. The information processing device 10 includes a processor 11, a main storage unit 12, an auxiliary storage unit 13, an operation unit 14, a display unit 15, a camera 16, a microphone 17, and a communication unit 18. The processor 11 reads out a program stored in the auxiliary storage unit 13 to the main storage unit 12 and executes the program, thereby realizing functions as functional configurations described in FIG. 2.

The operation unit 14 is, for example, a keyboard, a mouse, or a touch panel, and receives operations from a user. The display unit 15 is a display or the like. The camera 16 is a camera for capturing an image of the user in front of the display, such as a web camera or an in-camera. The microphone 17 collects a voice of the user and the like. The communication unit 18 is an interface for wired or wireless communication.

The information processing device 10 is a general-purpose computer such as a personal computer, a server computer, a tablet terminal, or a smartphone. The information processing device 10 may be achieved, for example, by distributed computing by multiple computer devices, or some of functional units may be achieved by a cloud server. Some of the functional units of the information processing device 10 may be achieved by dedicated hardware devices such as FPGAs or ASICs.

### (Functional Configuration)

With reference to FIG. 2, a functional configuration of the information processing device 10 will be described. FIG. 2 is a diagram illustrating the functional configuration of the information processing device 10. The information processing device 10 includes an acquisition unit 101, a psychological state determination unit 102, a work information storage unit 103, a work content decision unit 104, and an output unit 105.

The acquisition unit 101 acquires information for determining psychology that is information for determining the psychological state of the user. The information for determining psychology is, for example, a captured image of a face because a psychological state can be estimated from a facial expression and the like. The psychological state determination unit 102 determines the psychological state of the user when photographing on the basis of the information for determining psychology. The psychological state determination unit 102 determines the psychological state of the user, for example, using a learned model generated by deep learning such as convolution neural network (CNN) using the captured images of the face and the psychological states when photographing as training data. The acquisition unit 101 and the psychological state determination unit 102 are examples of an “acquisition unit” and a “determination unit”, respectively.

The work information storage unit 103 is a storage unit that stores, for each user, a past psychological state in association with information on work content and an amount of work (work information) performed in this past psychological state. The work information storage unit 103 stores and accumulates the psychological state of the user determined by the psychological state determination unit 102 and the work content and the amount of work actually performed by the user in this psychological state.

The work content and the amount of work actually performed by the user may be input by the user himself or herself using the operation unit 14, or may be transmitted from a work terminal when the work content and the amount of work can be acquired and measured by the work terminal or the like. Since the amount of work may change even in the same psychological state, the information processing device may store an average value of the amounts of work performed most recently in the same psychological state for a predetermined number of times (e.g., five times) as the amount of work in this psychological state. The work information storage unit 103 is an example of a “storage unit”.

The work content decision unit 104 acquires, from the work information storage unit 103, work information associated with a past psychological state that is the same as the current psychological state of the user determined by the psychological state determination unit 102. The work content decision unit 104 decides work content to be presented to the user on the basis of the acquired work information. The work content decision unit 104 is an example of a “decision unit”.

The output unit 105 presents the work content to be presented decided by the work content decision unit 104 to the user by displaying the work content to be presented on the display unit 15. The output unit 105 causes the work information storage unit 103 to store the psychological state of the user determined by the psychological state determination unit 102 in association with the work information including the work content to be presented decided by the work content decision unit 104 and the amount of work performed by the user. The output unit 105 is an example of a “presentation unit” and an “output unit”.

The above-described functions will be described as being enabled by one computer, but the functions may be achieved by multiple computers. For example, in a situation such as remote work, the acquisition unit 101 of the information processing device 10 may receive the information for determining psychology acquired by a work terminal of the user via the communication unit 18. The output unit 105 may transmit the work content to be presented decided by the work content decision unit 104 to the work terminal of the user via the communication unit 18 and cause the work terminal to display the work content to be presented.

### (Flow of Work Presentation Processing)

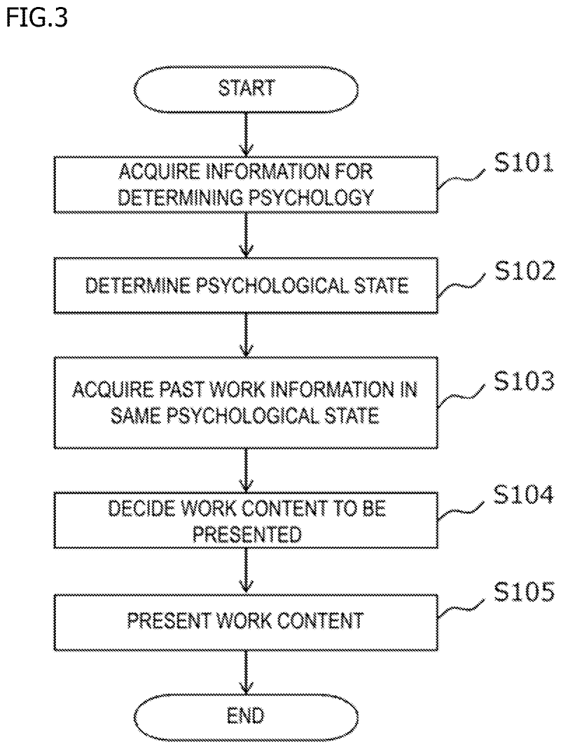

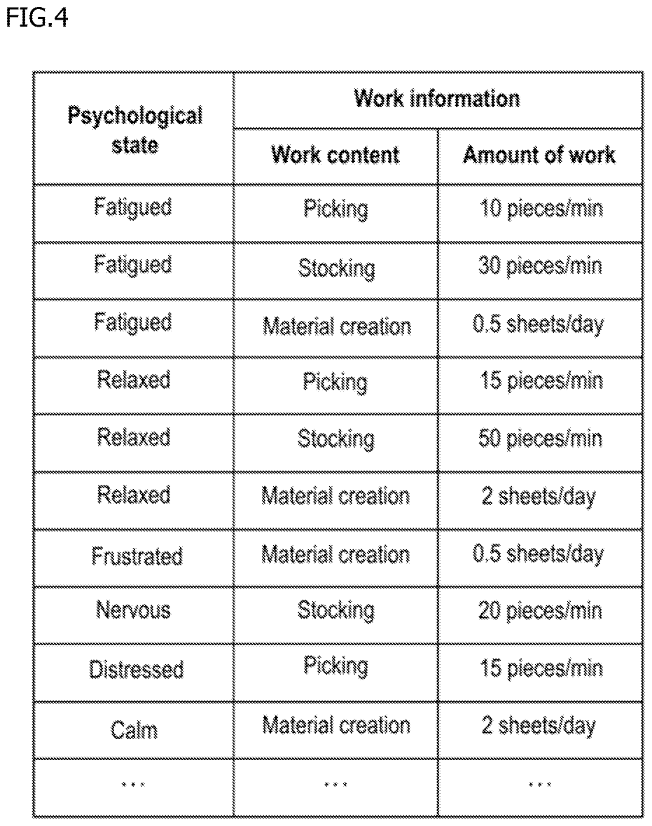

With reference to FIG. 3, a flow of work presentation processing according to a first embodiment will be described. The work presentation processing illustrated in FIG. 4 is started, for example, when a user who wants to start work turns his or her face to the display (the display unit 15).

In S101, the acquisition unit 101 acquires the information for determining psychology. The information for determining psychology is, for example, a captured image of the face of the user, which is captured by the camera 16. In S102, the psychological state determination unit 102 determines the psychological state of the user on the basis of the captured image of the face of the user. The psychological state determination unit 102 can determine the psychological state of the user by identifying the face of the user from a captured image and inputting this captured image into the learned model generated using the captured images of the face and the psychological states when photographing as training data. The captured images of the face that serve as the training data may be captured images of the face of the user himself or herself or may be captured images of a face of another user.

In S103, the work content decision unit 104 acquires the past work information associated with the past psychological state that is the same as the psychological state of the user determined in S102 from the work information storage unit 103.

Here, with reference to FIG. 4, the past psychological state and the past work information stored in the work information storage unit 103 will be described. FIG. 4 is a diagram illustrating data in the work information storage unit according to the first embodiment. The psychological states are predefined psychological states such as, but not limited to, fatigued, relaxed, frustrated, nervous, distressed, and calm.

The work information includes information on work content and an amount of work. The work content can be defined in advance by a manager. The amount of work can be an amount of work per unit time. The unit time may be specified in days, hours, or minutes. The unit time may be specified differently for different pieces of work. The psychological state and the work information illustrated in FIG. 4 are stored in the work information storage unit 103 for each user.

In S104, the work content decision unit 104 decides the work content to be presented to the user on the basis of the past work information acquired in S103. For example, when the psychological state of the user is determined to be “fatigued” in S102, the work content decision unit 104 acquires the pieces of work information for “picking”, “stocking”, and “material creation” in the example in FIG. 4. When multiple pieces of work information exist, the work content decision unit 104, for example, may decide the work content to be presented on the basis of the work information acquired first, or may decide the work content to be presented on the basis of preset priorities from the multiple pieces of work information.

Note that when multiple pieces of work information exist for one psychological state, the work content decision unit 104 may decide work content to be presented by combining the multiple pieces of work content. Alternatively, the work content decision unit 104 may determine a priority of the work content on the basis of skills of the user registered in advance, the total remaining amount of work, or the like, and may decide the work content to be presented on the basis of the priority. Alternatively, the work content decision unit 104 may decide, as work to be presented to the user, work in which a change in the amount of work due to a change in the psychological state is smaller than that in other pieces of work.

In S105, the output unit 105 presents the work content to be presented decided in S104 to the user by displaying the work content to be presented on the display. The output unit 105 may display the amount of work performed in the past as a reference of the amount of work together with the work content to be presented.

In the first embodiment, the information processing device 10 determines the current psychological state of the user on the basis of the captured image of the face, and decides the work content to be performed by the user on the basis of the past work content and the amount of work performed in the same psychological state as the current psychological state. Thus, the information processing device 10 can present the work in consideration of the psychological state of the worker and adjust workload of the worker.

### Second Embodiment

In the first embodiment, the information processing device 10 decides the work content to be presented on the basis of the past work information in the same psychological state as the current psychological state of the user. In a second embodiment, an information processing device 10 decides work content to be presented on the basis of whether a psychological state after performing work in the same psychological state as a current psychological state improved.

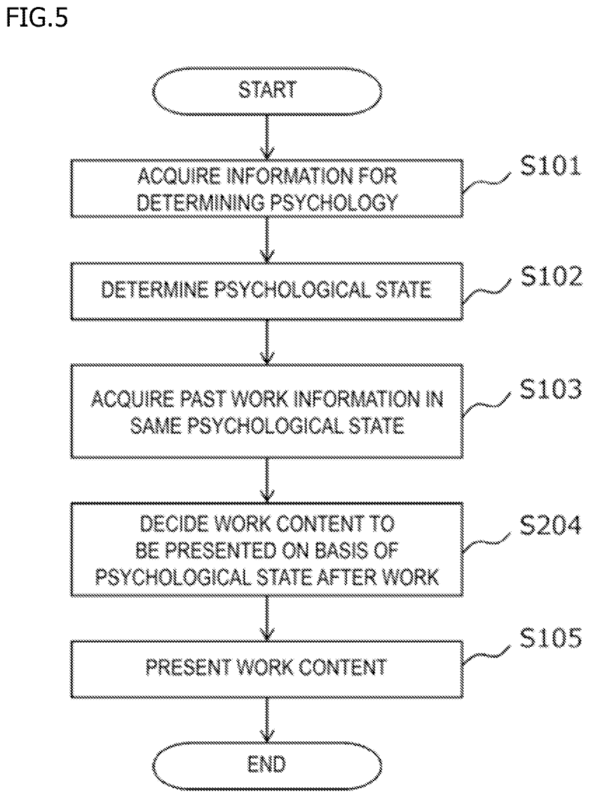

A hardware configuration and a functional configuration of the information processing device 10 according to the second embodiment are the same as those of the information processing device 10 according to the first embodiment, so description thereof will be omitted. FIG. 5 is a flowchart illustrating work presentation processing according to the second embodiment. Pieces of processing that are the same as those in the work presentation processing (first embodiment) illustrated in FIG. 3 are denoted by the same symbols, and description thereof will be omitted.

In S204, a work content decision unit 104 decides the work content to be presented to a user on the basis of a post-work psychological state. In the second embodiment, it is assumed that a work information storage unit 103 stores the psychological state of the user after actually performing the work, as one piece of work information associated with the psychological state.

The post-work psychological state can be determined at a timing when the user faces a display (a display unit 15), for example, immediately after the work or before starting work the next day, and can be stored in the work information storage unit 103. Note that for work performed while viewing the display, a psychological state determination unit 102 may determine the psychological state of the user during the work, and when there is a change, store the psychological state after the change in the work information storage unit 103.

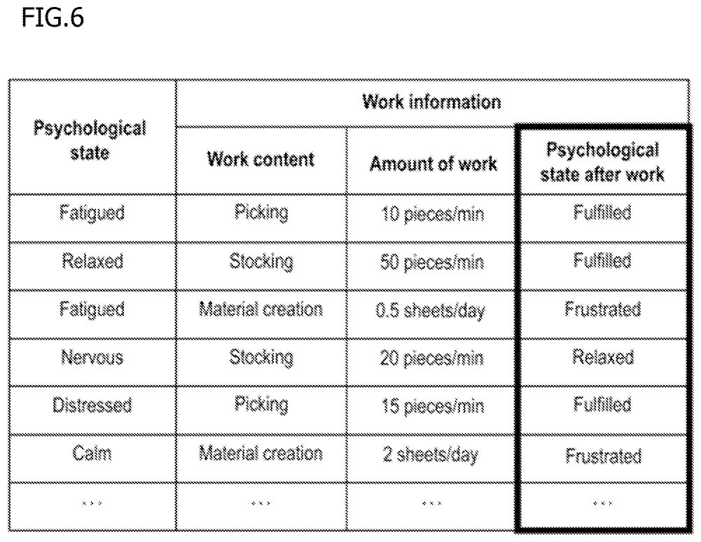

Here, with reference to FIG. 6, a past psychological state and past work information stored in the work information storage unit 103 will be described. FIG. 6 is a diagram illustrating data in the work information storage unit according to the second embodiment. In the second embodiment, the work information includes information on the post-work psychological state, in addition to work content and an amount of work. Items other than the post-work psychological state (psychological state, work content, and amount of work) are the same as those in FIG. 4, and thus description thereof will be omitted.

In the example in FIG. 6, when the psychological state was “fatigued”, after “picking”, the psychological state changed to “fulfilled”. When the psychological state was “fatigued”, after “material creation”, the psychological state changed to “frustrated”. The work information storage unit 103 stores a pre-work psychological state in association with the post-work psychological state. Thus, the work content decision unit 104 can determine whether the psychological state improved after work compared to before work.

Whether the post-work psychological state improved can be determined, for example, as follows. The psychological states were classified into positive and negative groups in advance, and when a psychological state in the negative group changed to a psychological state in the positive group, the work content decision unit 104 can determine that the psychological state improved. For example, the psychological states in the negative group are “fatigued”, “frustrated”, “nervous”, and “distressed”, and the psychological states in the positive group are “relaxed”, “calm”, and “fulfilled”.

In the example in FIG. 6, when the user performed “picking” in the psychological state of “fatigued”, the post-work psychological state was “fulfilled”. Since the psychological state changed from “fatigued” in the negative group to “fulfilled” in the positive group, the psychological state improved. On the other hand, when the user performed “material creation” in the psychological state of “fatigued”, the post-work psychological state was “frustrated”. Since the psychological state changed from “fatigued” in the negative group to “frustrated” in the negative group, the psychological state did not improve.

Note that determination of whether the psychological state improved is not limited to classifying the psychological states into two groups, but may be made by classifying the psychological states into three or more groups. Alternatively, a score is set for each psychological state of the user, and whether the psychological state of the user improves is determined in accordance with a change in the score. The work content decision unit 104 can determine that the psychological state improved when the score of the psychological state increased after work.

The work content decision unit 104 decides the work content to be presented, for example, as follows. When the current psychological state of the user is “fatigued”, in the example in FIG. 6, the work content decision unit 104 acquires two pieces of work information of “picking” and “material creation” from the work information storage unit 103 as candidates for the work content to be presented. The psychological state of the user improved to “fulfilled” after the work of “picking”, but the psychological state became “frustrated” after the work of “material creation” and did not improve. In this case, the work content decision unit 104 can decide “picking” through which the psychological state improved as the work content to be presented.

Alternatively, when the multiple pieces of work information exist for one psychological state, the work content decision unit 104 may decide the work content to be presented on the basis of a degree of improvement in the psychological state of the user. The degree of improvement in the psychological state, for example, when a score is set for each psychological state, can be determined to be a score obtained by subtracting a score of the pre-work psychological state from a score of the post-work psychological state. The work content decision unit 104 decides work content with a greater degree of improvement in the post-work psychological state as the work content to be presented.

Alternatively, when the multiple pieces of work information exist for one psychological state, the work content decision unit 104 may decide the work content to be presented on the basis of reliability of the determination for the psychological state of the user after improvement. The reliability here is reliability when determining the psychological state of the user after work using a predetermined learned model corresponding to the acquired information for determining psychology. The work content decision unit 104 decides work content having higher reliability for the determination of the psychological state of the user after work as the work content to be presented.

In the second embodiment, the information processing device 10 decides the work content to be presented to the user on the basis of the psychological state after performing the work. The work through which the psychological state improved after the work is presented with priority, making the work more efficient and allowing the worker to work more comfortably.

### Third Embodiment

In the second embodiment, the information processing device 10 decides the work content to be presented on the basis of the psychological state after performing the same work in the past in the same psychological state as the current psychological state. In a third embodiment, an information processing device 10 decides work content to be presented on the basis of a psychological state after the same work content was presented in the past in the same psychological state as a current psychological state.

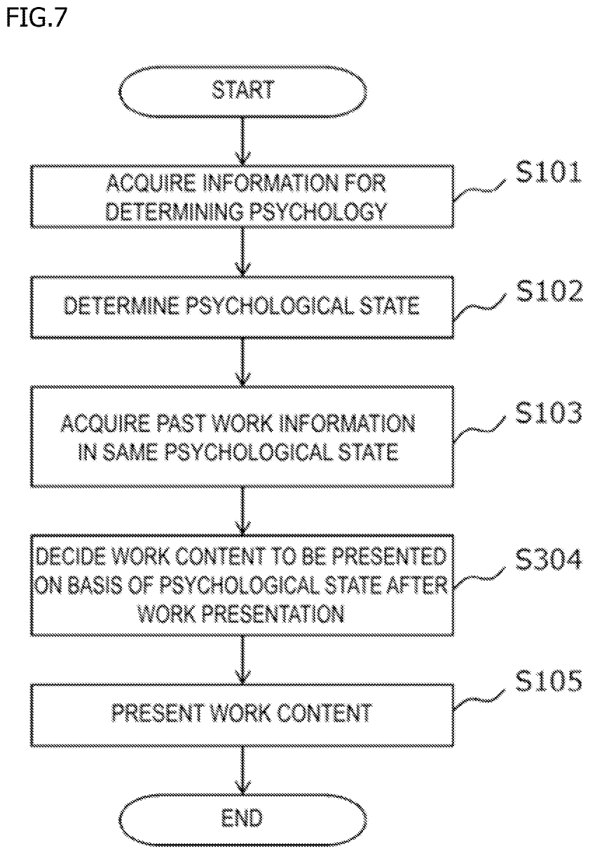

A hardware configuration and a functional configuration of the information processing device 10 according to the third embodiment are the same as those of the information processing device 10 according to the first embodiment, so description thereof will be omitted. FIG. 7 is a flowchart illustrating work presentation processing according to the third embodiment. Pieces of processing that are the same as those in the work presentation processing (first embodiment) illustrated in FIG. 3 are denoted by the same symbols, and description thereof will be omitted.

In S304, a work content decision unit 104 decides the work content to be presented to the user on the basis of the psychological state after work presentation. In the third embodiment, it is assumed that a work information storage unit 103 stores the psychological state of the user after being presented with the work content, as one piece of work information associated with the psychological state.

The psychological state after work presentation is the psychological state after the work content is presented. The psychological state after work presentation can be determined, for example, from a captured image of a face of the user after the work content is presented, and stored in the work information storage unit 103.

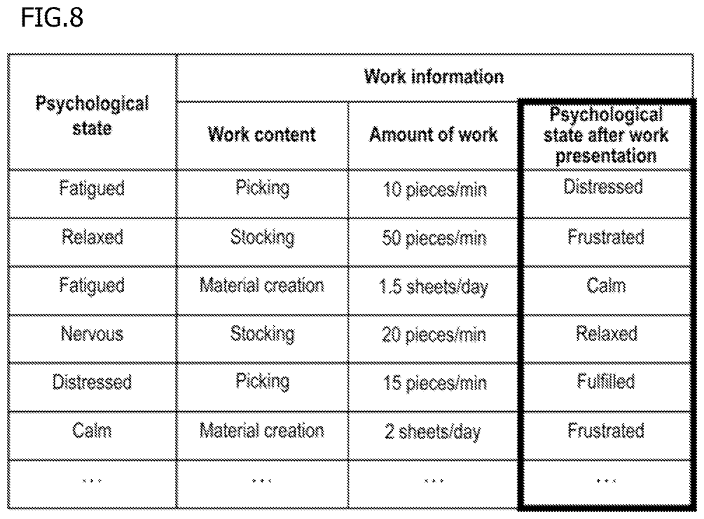

Here, with reference to FIG. 8, a past psychological state and past work information stored in the work information storage unit 103 will be described. FIG. 8 is a diagram illustrating data in the work information storage unit according to the third embodiment. In the third embodiment, the work information includes information on the psychological state after work presentation, in addition to work content and an amount of work. Items other than the psychological state after work presentation (psychological state, work content, and amount of work) are the same as those in FIG. 4, and thus description thereof will be omitted.

In the example in FIG. 8, when the psychological state was “fatigued” and “picking” was presented, the psychological state after work presentation was “distressed”. When the psychological state was “fatigued”, after the material creation was presented, the psychological state changed to “calm”. The work information storage unit 103 stores the psychological state before the work content was presented in association with the psychological state after the work content was presented. Thus, the work content decision unit 104 can determine whether the psychological state improved after work presentation compared to before work presentation. Whether the psychological state improved can be determined in the same manner as in the second embodiment.

The work content decision unit 104 decides the work content to be presented, for example, as follows. When the current psychological state of the user is “fatigued”, in the example in FIG. 8, the work content decision unit 104 acquires two pieces of work information of “picking” and “material creation” from the work information storage unit 103 as candidates for the work content to be presented. The psychological state of the user was “distressed” after work presentation of “picking” and did not improve, but improved to “calm” after work presentation of “material creation”. In this case, the work content decision unit 104 can decide “material creation” that improved the psychological state as the work content to be presented.

Alternatively, when the multiple pieces of work information exist for one psychological state, the work content decision unit 104 may decide the work content to be presented on the basis of a degree of improvement in the psychological state of the user. The degree of improvement in the psychological state of the user, for example, when a score is set for each psychological state, can be determined to be a score obtained by subtracting a score of the psychological state before work presentation from a score of the psychological state after work presentation. The work content decision unit 104 decides work content with a greater degree of improvement in the psychological state after work presentation as the work content to be presented.

Alternatively, when the multiple pieces of work information exist for one psychological state, the work content decision unit 104 may decide the work content to be presented on the basis of reliability of the determination for the psychological state of the user after improvement. The reliability here is reliability when determining the psychological state of the user after work presentation using a predetermined learned model. The work content decision unit 104 decides work content having higher reliability for the determination of the psychological state of the user after work presentation as the work content to be presented.

In the third embodiment, the information processing device 10 decides the work content to be presented to the user on the basis of the psychological state after presenting the work. The work that improved the psychological state after the work content was presented in the past is presented with priority, making the work more efficient and allowing the worker to work more comfortably.

### Fourth Embodiment

A fourth embodiment is an embodiment relating to various variations of acquisition of information for determining psychology and a method of determining a psychological state. Each variation can be implemented in combination with each of the first to third embodiments. A hardware configuration and a functional configuration of an information processing device 10 according to the fourth embodiment are the same as those of the information processing device 10 according to the first embodiment, so description thereof will be omitted.

### (Variation 4-1)

Variation 4-1 is an example in which a user is identified using a face recognition technique, and a psychological state of a user is determined using a learned model trained using captured images of a face of the identified user and psychological states when photographing as training data. By using the learned model trained using the captured images of the user himself or herself, the information processing device 10 can more accurately determine the psychological state of the user.

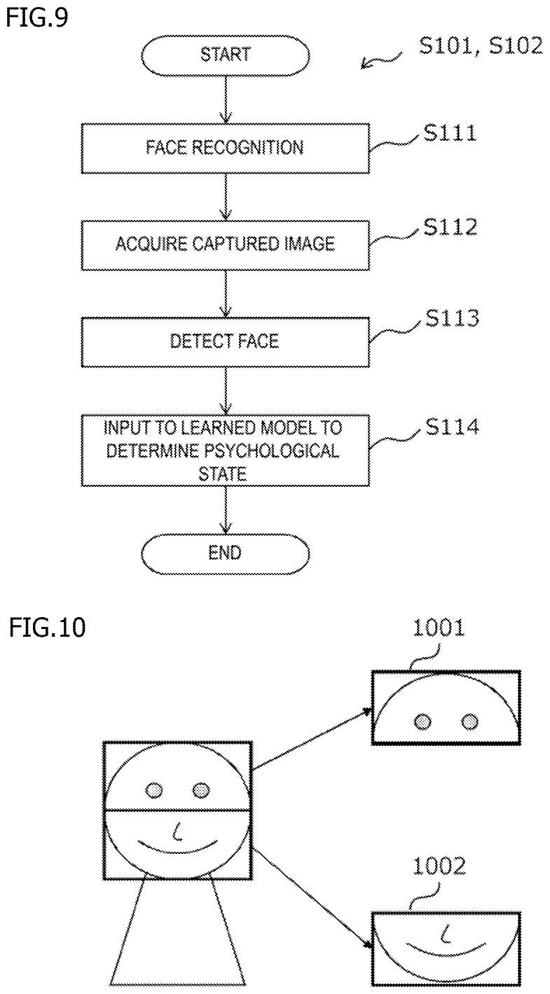

FIG. 9 is a flowchart illustrating processing to acquire information for determining psychology according to Variation 4-1. Pieces of processing illustrated in FIG. 9 are specific pieces of processing of the pieces of processing in S101 and S102 in FIGS. 3, 5, and 7.

In S111, an acquisition unit 101 identifies the user using a known face recognition technique. In S112, the acquisition unit 101 acquires a captured image of the user. The acquisition unit 101 may use an image acquired for face recognition in S111 as the captured image to be acquired in S112. In S113, the acquisition unit 101 detects the face of the user from the captured image acquired in S112 and acquires an image of the detected face as information for determining psychology.

In S114, a psychological state determination unit 102 inputs the image of the face detected in S113 to a learned model trained in advance using the captured images of the face of the user identified in S111 and the psychological states when photographing as training data. The psychological state determination unit 102 can determine the psychological state of the user from an output result of the learned model.

### (Variation 4-2)

Variation 4-2 is an example in which the psychological state of the user is determined using a learned model trained using captured images of parts of the face and the psychological states when photographing as training data. By making the determination on the basis of the captured images of the parts of the face in which the psychological state is likely to be expressed, the information processing device 10 can capture a subtle change in a facial expression and more accurately determine the psychological state of the user.

With reference to FIG. 10, parts of the face will be described. The parts of the face are, for example, an upper face region 1001 including eyes and a lower face region 1002 including a nose and a mouth. For a user whose psychological state tends to change easily in the upper face region 1001, the psychological state may be determined using the captured image in the region 1001, and for a user whose psychological state tends to change easily in the lower face region 1002, the psychological state may be determined using the captured image in the region 1002.

Note that the parts of the face are not limited to the upper face region 1001 and the lower face region 1002, but may be facial parts such as eyes, a nose, and a mouth. Alternatively, in combination with Variation 4-1, the information processing device 10 may determine the psychological state of an identified user using a learned model trained using captured images of parts of the face of the user and the psychological states when photographing as training data.

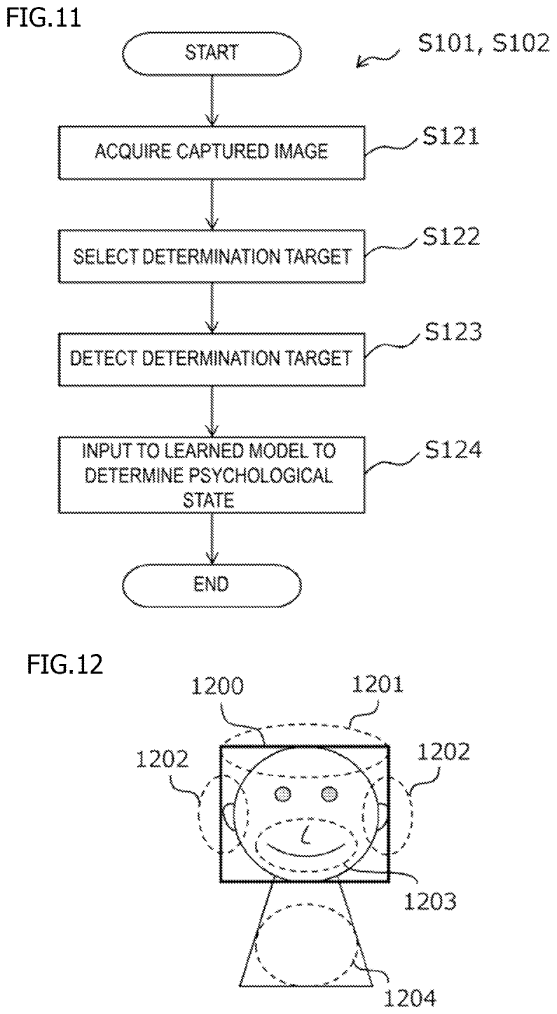

FIG. 11 is a flowchart illustrating processing to acquire information for determining psychology according to Variation 4-2. Pieces of processing illustrated in FIG. 11 are specific pieces of processing of the pieces of processing in S101 and S102 in FIGS. 3, 5, and 7.

In S121, the acquisition unit 101 acquires captured images of the user. In S122, the acquisition unit 101 selects a target for determination (a part of the face) for determining the psychological state. The target for determination is, for example, the upper face region 1001 or the lower face region 1002 described in FIG. 10. Alternatively, the target for determination may be the facial part such as eyes, a nose, or a mouth. For example, the acquisition unit 101 can select a region or a facial part preset for each user as a target for determination. Alternatively, the acquisition unit 101 may select a target for determination on the basis of an operation by the user. In S123, the acquisition unit 101 detects the target for determination selected in S122 from the captured image acquired in S121, and acquires an image of the detected part of the face as the information for determining psychology.

In S124, the psychological state determination unit 102 inputs the image of the part of the face detected in S123 to a learned model trained in advance using captured images of the part of the face and psychological states when photographing as training data. The psychological state determination unit 102 can determine the psychological state of the user from an output result of the learned model.

### (Variation 4-3)

Variation 4-3 is an example in which a psychological state of a user is determined using a learned model trained using pieces of information indicating a positional relationship between a face and a hand and psychological states when photographing as training data. The information processing device 10 can more accurately determine the psychological state of the user on the basis of a habit of the user indicated by the positional relationship between the face and the hand.

With reference to FIG. 12, the positional relationship between the face and the hand will be described. A rectangle 1200 surrounds a face part detected from the captured image. The hand of the user who has a habit of scratching or touching his or her head with his or her hand is detected in a head region 1201. The hand of the user who has a habit of scratching or touching his or her ear with his or her hand is detected in a region 1202 on the left and right of the face. The hand of the user who has a habit of placing his or her hand on his or her mouth or nose or touching his or her mouth or nose with his or her hand is detected in a lower face region 1203. The hand of the user who has a habit of placing his or her hand near his or her heart is detected in a chest region 1204.

Note that the information processing device 10 may capture video for a predetermined time (e.g., about 5 minutes to 10 minutes) and determine the psychological state of the user on the basis of a frequency with which the user moves his or her hand to each region described in FIG. 12 within the predetermined time instead of the position of the hand in the captured image. In this case, the information processing device 10 may determine the psychological state of the user using a learned model trained also using the frequency of moving the hand to each region within the predetermined time as input data.

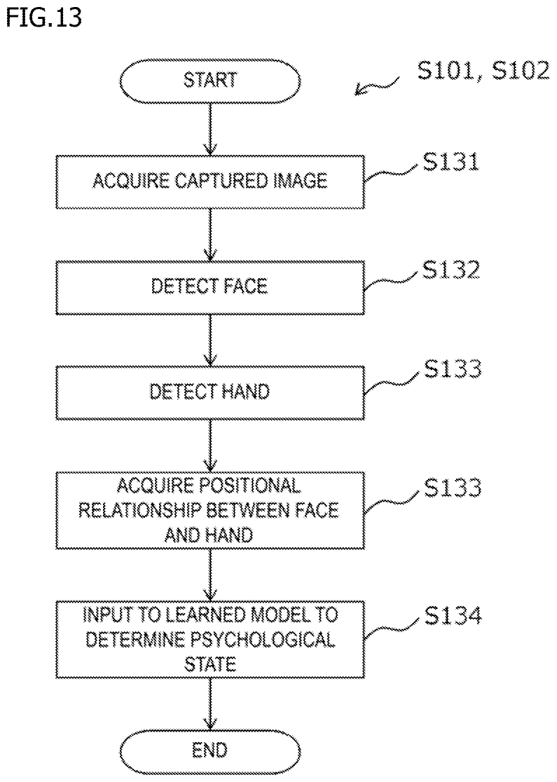

FIG. 13 is a flowchart illustrating processing to acquire information for determining psychology according to Variation 4-3. Pieces of processing illustrated in FIG. 13 are specific pieces of processing of the pieces of processing in S101 and S102 in FIGS. 3, 5, and 7.

In S131, the acquisition unit 101 acquires a captured image of the user. In S132, the acquisition unit 101 detects the face of the user from the captured image acquired in S131. In S133, the acquisition unit 101 detects the hand of the user from the captured image acquired in S131.

Note that when acquiring the captured image of the user in S131, the acquisition unit 101 may display a frame indicating a position of the face on the display and prompt the user to fit the face within the frame. By acquiring the captured image of the user so that the face of the user fits within the frame, the acquisition unit 101 can omit the processing to detect the face in S132.

In S133, the acquisition unit 101 acquires information indicating the positional relationship between the face and the hand as information for determining psychology. For example, the acquisition unit 101 can acquire the region in which the hand of the user is detected among the regions illustrated in FIG. 12 as the positional relationship between the face and the hand. The information indicating the positional relationship between the face and the hand may also include information such as a captured image of the face and the hand, a ratio of a region in which a partial region of the face and the hand overlap to an original region, and a frequency with which the hand overlaps the partial region of the face.

In S134, the psychological state determination unit 102 inputs the information indicating the positional relationship between the face and the hand acquired in S133 to the learned model trained in advance using the pieces of information indicating the positional relationship between the face and the hand and the psychological states when photographing as training data. The psychological state determination unit 102 can determine the psychological state of the user from an output result of the learned model.

### (Variation 4-4)

Variation 4-4 is an example in which the psychological state of the user is determined using a learned model trained using voice data of a voice of the user and the psychological states when recording as training data. For example, the information processing device 10 asks the user a question and prompts the user to speak. The learned model can determine the psychological state of the user by learning data such as voice tremor or voice pitch and corresponding psychological states. Even when the information processing device 10 is not provided with a camera 16, the information processing device 10 can determine the psychological state of the user on the basis of the voice data of the voice of the user.

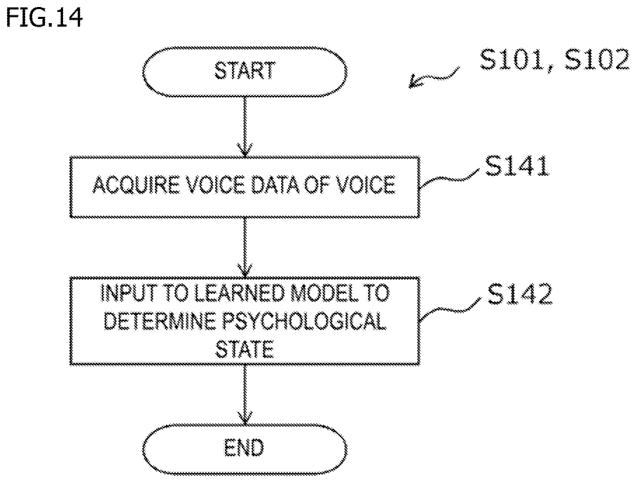

FIG. 14 is a flowchart illustrating processing to acquire information for determining psychology according to Variation 4-4. Pieces of processing illustrated in FIG. 14 are specific pieces of processing of the pieces of processing of S101 and S102 in FIGS. 3, 5, and 7.

In S141, the acquisition unit 101 acquires the voice data of the user as the information for determining psychology. In S142, the psychological state determination unit 102 inputs the voice data acquired in S141 to the learned model trained in advance using the voice data and the psychological states when recording as training data. The psychological state determination unit 102 can determine the psychological state of the user from an output result of the learned model.

### (Variation 4-5)

Variation 4-5 is an example in which a questionnaire survey is conducted to determine the psychological state of the user and the psychological state of the user is determined on the basis of answers of the user to the questionnaire. Even when it is difficult to determine the psychological state from the facial expression or the tone of the voice, the psychological state of the user can be determined more accurately by appropriately setting questions and answer options in the questionnaire.

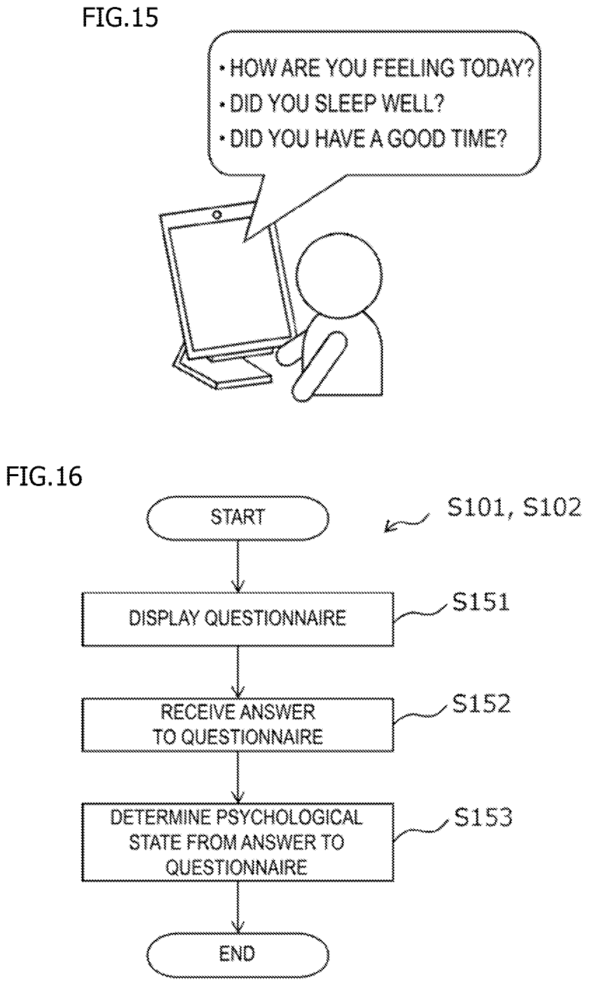

With reference to FIG. 15, the questionnaire will be described. Questions in the questionnaire are to be questions that are easy to estimate the psychological state, such as “How are you feeling today?”, “Did you sleep well?”, and “Did you have a good time?”. The information processing device 10 may display the next question in accordance with an answer of the user to the question in the questionnaire. For example, the questions in the questionnaire can be a group of questions in which the next question is selected on the basis of the answer of the user to narrow down the candidate psychological states.

FIG. 16 is a flowchart illustrating processing to acquire information for determining psychology according to Variation 4-5. Pieces of processing illustrated in FIG. 16 are specific pieces of processing of the pieces of processing in S101 and S102 in FIGS. 3, 5, and 7.

In S151, the acquisition unit 101 displays questions in the questionnaire for determining the psychological state of the user. In S152, the acquisition unit 101 receives answers of the user to the questionnaire. In S153, the psychological state determination unit 102 can determine the psychological state of the user from the answers of the user to the questionnaire.

### (Others)

The psychological state determination unit 102 may combine multiple variations among the above-described variations to determine the psychological state of the user using multiple pieces of information for determining psychology. In this case, the psychological state determination unit 102 can determine, as the psychological state of the user, a psychological state having the highest reliability among the psychological states determined using the corresponding learned models from the pieces of information for determining psychology.

In the fourth embodiment, the information processing device 10 can more accurately determine the psychological state of the user by acquiring various pieces of information for determining the psychological state (information for determining psychology) from the user. The determination of the psychological state of the user is performed, for example, before work, after work presentation, or after performing work. Note that the pre-work psychological state may be determined each time the work content changes. The determination of the psychological state after performing work is not limited immediately after performing work, but may be before starting work the next day. The determination of the psychological state after performing work is not limited after completing work, but may be during work.

In addition, the determination of the psychological state of the user is not limited to being based on the information for determining psychology acquired at a specific time point, but may be based on, for example, information for determining psychology acquired during a predetermined period of time (e.g., a captured video during a predetermined period of time).

### <Other>

The above-described embodiments and variations are merely illustrative configuration examples of the present invention. The configurations of the embodiments and the variations are not limited to the specific forms described above, and can be used in combined as appropriate within the scope of the technical idea of the present invention. The present invention can be varied in various ways without departing from the technical idea thereof.

### <Supplementary Note>

(1) An information processing device (10) includes an acquisition unit (101) configured to acquire information for determining psychology as being information for determining a psychological state of a user, a determination unit (102) configured to determine the psychological state of the user on the basis of the information for determining psychology, a storage unit (103) configured to store past psychological states of the user in association with pieces of work information, each of the pieces of work information including work content and an amount of work of the user in each of the past psychological states, and a decision unit (104) configured to acquire work information associated with a past psychological state being the same as a current psychological state of the user from the storage unit and decide work content to be presented to the user on the basis of the work information acquired.

(2) An information processing method executed by a computer (10), the information processing method includes an acquiring step (S101) of acquiring information for determining psychology as being information for determining a psychological state of a user, a determination step (S102) of determining the psychological state of the user on the basis of the information for determining psychology, an acquiring step (S103) of acquiring, from a storage unit configured to store past psychological states of the user in association with pieces of work information, each of the pieces of work information including work content and an amount of work of the user in each of the past psychological states, work information associated with a past psychological state being the same as a current psychological state of the user, and a deciding step (S104) of deciding work content to be presented to the user on the basis of the work information acquired.

### REFERENCE SIGNS LIST

1: information processing device, 11: processor, 12: main storage unit, 13: auxiliary storage unit, 14: operation unit, 15: display unit, 16: camera, 17: microphone, 18: communication unit 101: acquisition unit, 102: psychological state determination unit, 103: work information storage unit, 104: work content decision unit, 105: output unit

1. An information processing device comprising:
an acquisition unit configured to acquire information for determining psychology as being information for determining a psychological state of a user;
a determination unit configured to determine the psychological state of the user on the basis of the information for determining psychology;
a storage unit configured to store past psychological states of the user in association with pieces of work information, each of the pieces of work information including work content and an amount of work of the user in each of the past psychological states; and
a decision unit configured to acquire work information associated with a past psychological state being the same as a current psychological state of the user from the storage unit and decide work content to be presented to the user on the basis of the work information acquired.

2. The information processing device according to claim 1, wherein
each of the pieces of work information associated with the past psychological states further includes information on a post-work psychological state after the user performed work content of each of the pieces of work information, and
the decision unit decides the work content to be presented on the basis of work information in which the post-work psychological state is improved compared to a pre-work psychological state from pieces of work information associated with the past psychological state being the same as the current psychological state.

3. The information processing device according to claim 1, wherein
each of the pieces of work information associated with the past psychological states further includes information on a psychological state after work presentation when the user is presented with work content of each of the pieces of work information, and
the decision unit decides the work content to be presented on the basis of work information in which the psychological state after work presentation is improved compared to a psychological state before work presentation from pieces of work information associated with the past psychological state being the same as the current psychological state.

4. The information processing device according to claim 2, wherein
a score is set for a psychological state one by one for the psychological state of the user, and whether the psychological state of the user improved is determined in accordance with a change in the score.

5. The information processing device according to claim 2, wherein
when there are multiple pieces of work information associated with the past psychological state being the same as the current psychological state, the decision unit decides the work content to be presented on the basis of at least one of a degree of improvement in the psychological state of the user or reliability of determination of the psychological state of the user after improvement.

6. The information processing device according to claim 1, wherein
the information for determining psychology includes a captured image of a face or part of the face of the user, and
the determination unit determines the psychological state of the user using a learned model trained using the captured image of the face or the part of the face and a psychological state when photographing as training data.

7. The information processing device according to claim 1, wherein
the determination unit identifies the user by face recognition, and determines the psychological state of the user using a learned model trained using a captured image of a face of the user or part of the face of the user and a psychological state when photographing as training data.

8. The information processing device according to claim 7, wherein
the information for determining psychology includes information indicating a positional relationship between a face and a hand of the user, and
the determination unit determines the psychological state of the user using a learned model trained using the information indicating the positional relationship between the face and the hand of the user and a psychological state when photographing as training data.

9. The information processing device according to claim 6, wherein
the determination unit detects the face of the user from a captured image captured with the face positioned within a frame displayed on a display unit by the user.

10. The information processing device according to claim 1, wherein
the information for determining psychology includes voice data of a voice of the user, and
the determination unit determines the psychological state of the user using a learned model trained using the voice data of the voice of the user and a psychological state when recording as training data.

11. The information processing device according to claim 1, wherein
the information for determining psychology includes an answer of the user to a questionnaire for determining the psychological state of the user, and
the determination unit determines the psychological state of the user on the basis of the answer to the questionnaire.

12. The information processing device according to claim 1, wherein
when determining the psychological state of the user using multiple pieces of the information for determining psychology, the determination unit determines, as the psychological state of the user, a psychological state having the highest reliability from psychological states determined from the multiple pieces of the information for determining psychology.

13. The information processing device according to claim 1, further comprising:
a presentation unit configured to present the work content to be presented decided by the decision unit to the user.

14. The information processing device according to claim 1, further comprising:
an output unit configured to cause the storage unit to store the psychological state of the user determined by the determination unit in association with work information including the work content to be presented decided by the decision unit and an amount of work performed by the user.

15. An information processing method executed by a computer, the information processing method comprising:
acquiring information for determining psychology as being information for determining a psychological state of a user;
determining the psychological state of the user on the basis of the information for determining psychology;
acquiring, from a storage unit configured to store past psychological states of the user in association with pieces of work information, each of the pieces of work information including work content and an amount of work of the user in each of the past psychological states, work information associated with a past psychological state being the same as a current psychological state of the user; and
deciding work content to be presented to the user on the basis of the work information acquired.

16. A non-transitory computer readable medium storing a program for causing a computer to execute operations comprising:
acquiring information for determining psychology as being information for determining a psychological state of a user;
determining the psychological state of the user on the basis of the information for determining psychology;
acquiring, from a storage unit configured to store past psychological states of the user in association with pieces of work information, each of the pieces of work information including work content and an amount of work of the user in each of the past psychological states, work information associated with a past psychological state being the same as a current psychological state of the user; and
deciding work content to be presented to the user on the basis of the work information acquired.
